(19) United States
(12) Patent Application Publication
(10) Pub. No.: US 2007/0299307 A1
(43) Pub. Date: Dec. 27, 2007

(54) Disposable Syringe

(75) Inventors: Choong Keong Lew, Kuala Lumpur (MY); Poo Hook, Kuala Lumpur (MY)

Correspondence Address: BROOKS KUSHMAN P.C., 1000 TOWN CENTER, TWENTY-SECOND FLOOR, SOUTHFIELD, MI, 48075, US

(73) Assignee: DOLOMITE TECHNOLOGY SDN BHD, Kuala Lumpur (MY)

(21) Appl. No.: 11/570,004

(22) PCT Filed: Jun. 3, 2005
(86) PCT No.: PCT/SG05/00177

§ 371 Date: Jun. 14, 2007

(30) Foreign Application Priority Data
Jun. 4, 2004 (MY) PI 20042168

(51) Int. Cl. A61M 5/00; A61M 5/178; A61M 5/315

(52) U.S. Cl. 600/110; 604/228; 604/38

(57) ABSTRACT

A disposable syringe having a plunger (20) which is movable within a barrel (170), a needle carrier (80) to retain a needle (300), and, a connection means (70) attached to the plunger. In use, upon depression of the plunger (20), the connection means (70) engages the needle carrier (80). This is achieved such that there is angular displacement of the needle carrier (80) relative to the longitudinal axis of the syringe. Therefore, upon subsequent withdrawal of the plunger (20) the needle carrier (80) becomes housed in the barrel (170) in a canted manner.

### FIELD OF THE INVENTION

The present invention relates to a disposable syringe, and in particular, to a disposable syringe in which the needle becomes housed within the barrel of the syringe, after use.

### BACKGROUND OF THE INVENTION

The reference to any prior art in this specification is not, and should not be taken as, an acknowledgement or any form of suggestion that that prior art forms part of the common general knowledge in Australia.

With the increase in hazards due to needle-stick injuries and the safety issues concerned with syringes, there has been an increased need for the development of disposable syringes. There is also a need to have single-use syringes, which are not only environment friendly, but are also effective for medical use. This means that the quality of the disposable syringes should be of a high standard.

US 2003/0158525 describes a retractable syringe whereby the barrel of the syringe, engages the plunger with one or two projections. The document does not describe a syringe, which is retractable and leak proof, whereby the needle is kept securely in the barrel of the syringe.

DE 4321626 describes a locking disk for use in a syringe, whereby the locking disk is stopped by a stop ring, when the plunger is pulled for re-use of the syringe, thereby stopping re-use of the syringe. This document does not describe a locking mechanism which is separate to a sealing device. Nor does it describe a mechanism by which the needle is kept securely in the barrel of the syringe.

After the retraction of needle, the prior art devices generally prevent the needle coming out of the barrel from the proximal end of the barrel by breaking the plunger or jamming the plunger. Breaking of the plunger requires one additional step, which involves training and change of behaviour. Jamming of the plunger is not reliable because the plunger can still be pulled out of the barrel by force. When force is used, often the plunger and the needle carrier come out, leaving the needle free to come out as well. Sometimes, due to manufacturing limitations, it is also possible to reinsert the plunger for re-use of the syringe.

The present sealing devices in the prior art, have both quality and reliability issues. The sealing devices are generally also used as a locking device, which is a disadvantage, since retractable syringes generally require appropriate locking force and a good seal, which is difficult to achieve with the prior art. The prior art devices also have the limitation in that, during the manufacturing/assembly processes, variations in the diameter and the incline of the sleeve of the barrel, and the size, diameter and elasticity of the sealing device will generally result in various malfunctions of the needle.

For example, if the sealing device is much bigger than the interior surface of the sleeve, excessive interference force will lock the needle carrier permanently and cause the socket to slip at the head of the needle carrier during retraction, hence the needle cannot be retracted. If the sealing device is slightly larger than the interior surface of the sleeve, the locking force might not be strong enough to prevent needle movement during injection. If the sealing device is not pre-compressed at a pre-determined level, leaking may occur, with air bubbles going in during suction of the medication, and liquid leaking during depression of the plunger for injection. In the worst case, the needle carrier may be sucked inside the barrel during suction of the fluid.

Screening of the barrels and the sealing devices for the desired tolerances can prevent some potential malfunctions, however, these will increase the production cost tremendously.

Some malfunctions may also arise during the shelf life of the syringe, generally effecting older stock. The compressed sealing device may lose its elasticity and will therefore be unable to maintain its locking force. Hence, the product will fail prematurely before reaching the end of its shelf life, and will cause a disaster during an emergency situation, thereby causing a general health risk. Unpredictable quality and high risk of product recall are also problems faced by the industry.

Some prior art devices also cause leaks from the needle base. This occurs when the mating surface between the needle hub and the stem of needle carrier has an air gap resulting in air leaking during suction, and liquid leaking during depression of the plunger.

Most of the commercially available safety syringes are pre-packed with fixed needles. This means that needle sizes have to be determined before ordering. The uncertainties in size have increased the stock level of the plastic syringes since they come together with the needles. The need to obtain additional syringes is not commercially viable for medical institutions. The increase in stock holding cost and space will deter many medical professionals from early adoption of safety syringes.

Further, the design of disposable syringes and poor manufacturing quality is not conducive to the practitioner securing a needle onto the needle carrier. Given that the needle carrier is a separate piece from the barrel, often there is no traction to enable the needle to be screwed securely onto the needle carrier without restraining the carrier in some way. Methods of fixing needles securely to the needle carrier may also interfere with the method of retracting the needle into the barrel for disposal purposes.

Before retraction of needle, prior art generally teaches the use of the plunger to engage the needle carrier, to turn and unlock the needle carrier from the neck of the barrel. This unusual action requires training of the medical professionals to have effective change in behaviour. Another more natural way is using friction to lock the needle carrier. However, too high a friction force will transfer the shock action to the recipient of injection causing immediate pain and phobia in the near future.

It will be appreciated that there are various drawbacks associated with the prior art devices.

### SUMMARY OF THE INVENTION

The present invention seeks to provide a disposable syringe which overcomes some of the prior art.

The present invention also seeks to provide a disposable syringe in which the needle becomes safely housed in the barrel of the syringe, after use.

In one broad form, the present invention provides a disposable syringe, including:

a plunger movable within a barrel;

a needle carrier to retain a needle; and,

a connection means attached to the plunger,

whereby, upon depression of the plunger said connection means is adapted to engage the needle carrier in an angularly displaced manner, such that, upon subsequent withdrawal of the plunger, the needle carrier and needle become housed within the barrel in a canted manner.

Preferably, the connection means includes an engaging recess formed with a neck and an enlarged chamber, and, the needle carrier includes a plurality of hooks, each hook having a deformable arm and an enlarged head, such that, when said hooks become fully inserted within the engaging recess, the arms of the hooks spring back to their uncompressed state and the head of each hook is permanently retained within the chamber.

Preferably, said neck of said engaging recess of said connection means is angularly offset from a longitudinal axis of said syringe.

Preferably, said connection means is formed of rubber or like material to permit some deformation during engagement of the needle carrier therewith.

Preferably, each hook is formed of rigid by somewhat flexible plastics or like material, the permit some deformation during engagement with said connection means.

Preferably, said connection means is permanently attached to said plunger.

Preferably, said connection means is removably attached to said plunger.

Preferably, said connection means is attached to said plunger via a shaped protrusion being provided on said plunger which is adapted to be received in a corresponding recess provided in said connection means, or vice versa.

Preferably, the needle carrier includes a sealing device as a separate component.

Preferably, the sealing device includes a flange that is wider than the opening of the barrel, and is adapted to form a sealing between the outer wall of the sealing device and the inner wall of a barrel.

Preferably, the sealing device has a seat below the flange.

Preferably, the sealing device is formed as an O-ring.

Preferably, the sealing device has at least one chamfered edge.

Preferably, sealing device is chamfered at the lower side of the sealing device.

Preferably, the barrel has, shoulders at the distal end, which allows for the resting of the sealing device.

Preferably, the seat of the sealing device rests on the barrel shoulders.

Preferably, the shoulders are substantially flat with sloped edges.

Preferably, the shoulders are substantially near a thicker barrel wall.

Preferably, the barrel has at least one ratchet protrusion at its proximal end.

Preferably, the chamfered edge of the sealing device allows for the sealing device to pass through the ratchet protrusions of the barrel, upon entering the barrel at the proximal end, however, does not allow for the sealing device to exit the barrel.

Preferably, two or more protrusions are attached to anywhere along the interior barrel wall for engagement with the needle carrier.

Preferably, the protrusions have a pyramid-shaped head.

Preferably, the needle carrier is formed such that it includes depressions, which are able to receive the protrusions along the barrel wall and/or barrel sleeve, in order for the barrel to engage the needle carrier.

Preferably, the needle carrier is engaged by the barrel by a locking mechanism.

Preferably, the locking mechanism is a snap-lock mechanism.

Preferably, the needle carrier is adapted to hold the needle by the use of a carrier stem.

Preferably, the carrier stem is attached to a needle hub.

Preferably, the carrier stem and the needle hub are attached using the Luer-lock principle.

Preferably, the stem is formed by a truncated cone.

Preferably, the needle hub is formed by a truncated cone.

Preferably, the stem is inserted into the hub.

Preferably, a screw thread engages the stem and hub.

Preferably, a female thread engages the stem and hub.

Preferably, the needle hub is turned, thereby creating a seal between the hub and the stem such that the diameter of the stem is substantially larger than the diameter of the hub, and the stem and the hub are at the same angle.

Preferably, the hooks are of spring formation.

Preferably, the connection means is formed of rubber or plastic material.

Preferably, the needle carrier is formed of plastic material.

Preferably, once the needle has been retracted, it is adapted to rest any where along the barrel wall, whereby by further depression of the plunger the needle is bent.

Preferably, once the needle has been retracted, it is adapted to rest any at the shoulders of the barrel wall, whereby by further depression of the plunger the needle pushes the shoulder and is thereby bent.

Preferably, after bending of the needle, further depression of the plunger results in coiling of the needle.

In a further broad form, the present invention provides a connection means, for use in a syringe, adapted to be attached to a plunger, and to receive a needle carrier, the needle carrier being connected to a needle, such that, the connection means enables a substantial angular displacement of the needle, thereby canting the needle, after the retraction of the needle into the syringe.

Preferably, said connection means is formed of rubber or like material to permit some deformation during engagement of the needle carrier therewith.

Preferably, said connection means including an engaging recess formed with a neck and an enlarged chamber, for engagement with a plurality of hooks of said needle carrier, each hook having a deformable arm and an enlarged head, such that, when each hook is inserted within the engaging recess, the arm of the hooks spring back to their uncompressed state and the head of each hook is permanently retained within the chamber.

Preferably, the neck of said engaging recess of said connection means is angularly offset from a longitudinal axis of the syringe.

In a further broad form, the present invention provides a needle carrier, for use in a syringe, the needle carrier including:

at least one holding means;

a sealing device

whereby the needle carrier is adapted to be attached to a needle, and can also be attached to the plunger of a syringe, through the holding means, and whereby the sealing means is a separate component to the holding means.

Preferably, the needle carrier is adapted to be attached to a needle, through a stem.

Preferably, the needle holding means includes a plurality of hooks.

In a further broad form, the present invention provides a sealing device for use in a syringe, the sealing device including a flange, whereby the flange is adapted to be wider than the opening of the syringe, and when inserted into the syringe, the flange is compressed such that to allow sealing between the flange and the syringe.

### BRIEF DESCRIPTION OF THE DRAWINGS

The present invention will become more fully understood from the following detailed description of a preferred but non-limiting embodiment thereof, described in connection with the accompanying drawings, wherein:

FIG. 1 shows various elements of the syringe of the present invention, including the plunger, connection means, and the needle carrier;

FIG. 2 shows how the plunger may be attached to the connection means;

FIG. 3 shows the barrel, the needle hub as attached to the needle, and the cap, of the syringe of the present invention;

FIG. 4 shows various elements of the barrel and the needle hub, and needle as covered by the cap;

FIG. 5 shows the needle carrier inserted into the barrel;

FIG. 6 shows the barrel with the plunger, with the connection means attached, and needle carrier inserted, and the needle hub with needle and cap before connection to the needle carrier;

FIG. 7 shows the syringe prior to use, before removal of the needle cap;

FIG. 8 shows the syringe ready for injection, with plunger retracted with the suction of the fluid in the barrel, and the needle cap removed;

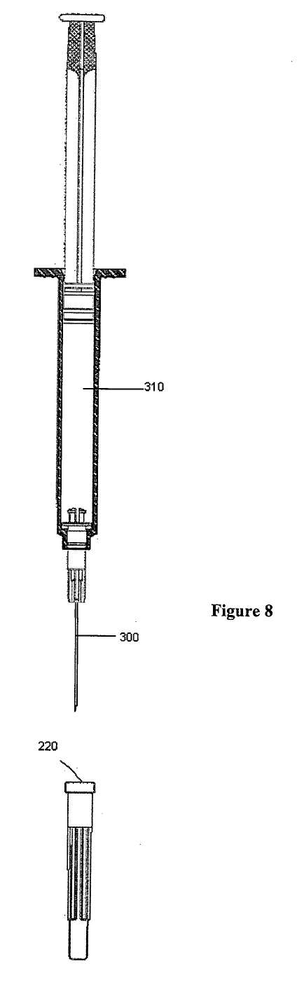

FIG. 9 shows the depression of the plunger completing the injection of the fluid, showing how the connection means is used to connect the needle carrier to the plunger;

FIG. 10 shows complete depression of the plunger, whereby the hooks of the needle carrier are engaged with the connection means;

FIG. 11 shows retraction of the plunger, which pulls the needle carrier and needle up into the barrel, and also, how canting of the needle occurs;

FIG. 12 shows depressing the plunger once more, whereby due to canting of the needle, the needle presses along the wall of the barrel and the needle is subsequently bent and coiled, thereby remaining inside the barrel, and unable to be re-used; and,

FIG. 13 shows excessive force is used to pull the plunger out of the barrel, how the flange of the sealing device catches onto the ratchet protrusions of the barrel, thereby retaining the coiled needle carrier and needle inside the barrel.

### DETAILED DESCRIPTION OF THE PREFERRED EMBODIMENT

Throughout the drawings, like numerals will be used to identify similar features, except where expressly otherwise indicated. Also, the following meanings are given to certain terms:

Plurality as used herein refers to two or more.

Proximal as used herein refers to the top of, or away from the needle.

Distal as used herein refers to the bottom of, or closer to the needle.

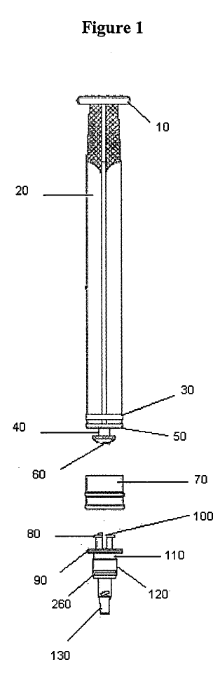

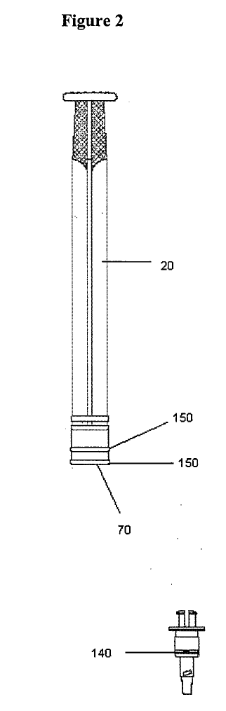

FIG. 1 shows preferred embodiments of certain components of the invention. The plunger 20 is shown, which has a plunger flange 10 at its proximal end, which allows for the depression of the plunger, and in one preferred embodiment, at its distal end, there exists a stopper flange 30 and a socket flange 50. The socket flange is attached to the socket neck 40, which is in turn attached to a socket head 60. The socket head is adapted to be attached to the connection means 70. In another preferred embodiment, the connection means can be attached to the plunger by gluing the connection means to the plunger, or by any other mechanical means, as will be apparent to persons skilled in the art. The plunger 20, as attached to the connection means 70 is further shown in FIG. 2. FIG. 2 also shows that in one preferred embodiment, the connection means can include piston rings 150.

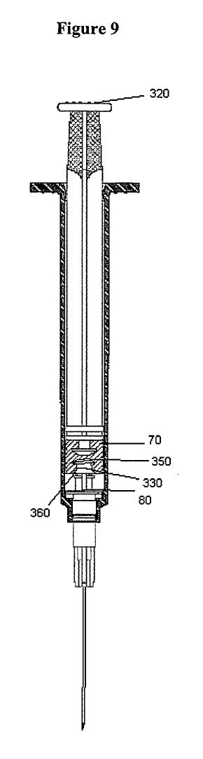

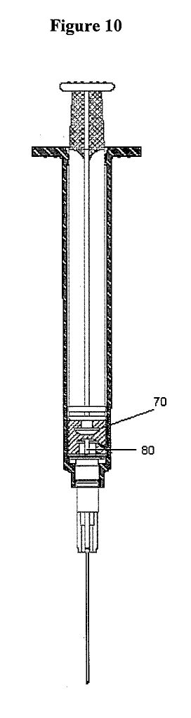

FIG. 1 also shows the needle carrier 120, which includes a plurality of hooks 80, which can include deflection slots 100. The connection means 70 is adapted to engage the needle carrier 120. FIG. 9 shows a preferred embodiment, whereby upon depression of the plunger 320 for injection, the hooks 80 of the needle carrier approach an engaging recess 330 of the connection means 70. The engaging recess includes a neck 360 and an enlarged chamber 350. The engaging recess is preferably naturally angularly displaced, and upon insertion of the hook 80, as shown in FIG. 10, into the engaging recess, the arms of the hooks spring back to their uncompressed state and the head of each hook is permanently retained within the chamber, thereby, deforming the engaging recess. Upon subsequent withdrawal of the plunger, the needle carrier 120 and the needle 300 become housed within the barrel and the engaging recess then reforms to its naturally angularly displaced position, such that the needle and the needle carrier are canted inside the barrel.

In one embodiment, FIG. 9 the neck 360 of the engaging recess is the same (or slightly smaller) size as the neck of the needle carrier 80. The longitudinal axis of the neck forms an acute angle with the longitudinal axis of the plunger. (Theoretically, the longitudinal axes of the plunger, the barrel, the needle carrier, and the needle or have the longitudinal axis coincide with each other.) The enlarged chamber of the engaging recess can be any shape (cylinder, truncated cone and so on) as long as it is bigger than the overall dimension of all the hooks combined.

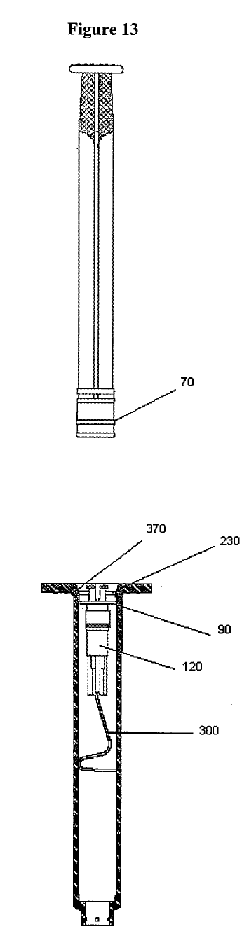

In another preferred embodiment, the invention uses spring like hooks and rubber-like catch for engagement of the needle carrier. In yet, another embodiment, the needle carrier head is spliced (two protrusions, secured at the bottom, flexible at the top). The protrusions can have arrow like heads and they can move towards each other, whereby the arrowheads act like a chamfer, and can engage with the bottom of the socket head. Once engaged, protrusions move away from each other, the bottom of the arrow heads hooking onto the inner lip of the socket head. This allows for the reduction of the engagement force drastically, and also has a positive catch when the hook opens up and is engaged by the connection means during retraction of the plunger. Reduced pressure on the plunger reduces pain, when needle carrier engages with the socket, and when it is retracted. This also allows detachment of the needle carrier when extraordinary force is used during retraction of the plunger, as shown in FIG. 13.

The needle carrier 120 further includes a sealing device, which is preferably separable from the hooks. In another embodiment, the sealing device is formed as an O-ring. As shown in FIG. 1, the sealing device can include a flange 90 and a seat 110.

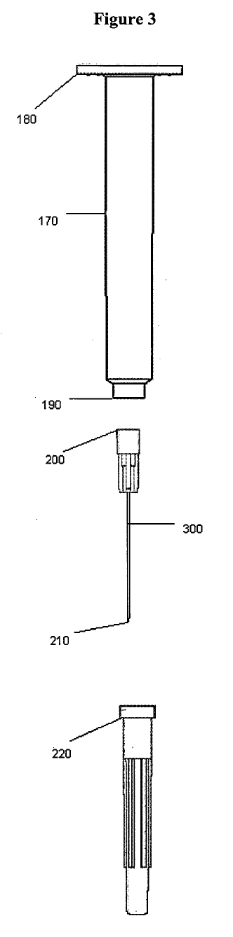

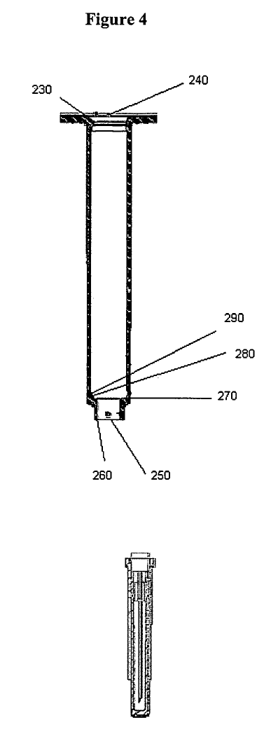

A preferred embodiment of the barrel 170 of the syringe is shown in FIG. 3. The barrel 170 includes a barrel holder 180 and a barrel sleeve 190. FIG. 4 further shows the preferred embodiment of the barrel, with the shouldered part of the barrel wall 270 at the distal end 250, and the ratchet protrusions 230 at the proximal end 240.

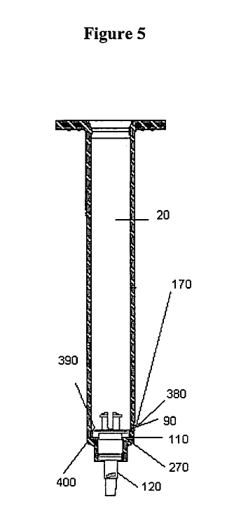

In another embodiment, the needle carrier is kept inside the barrel sleeve 190 (shown in FIG. 3), by a snap groove 140 (FIG. 2), whereby they are snap-locked 260 (FIG. 4). The needle carrier is also kept inside the barrel by resting the seat 110 of the sealing device (FIG. 5) on the barrel shoulder 270.

The O-ring flange 90 of the needle carrier 120 is slightly bigger/wider than opening of barrel at the proximal end 240. The corresponding chamfered edges on the O-ring flange permit needle carrier insertion into barrel during manufacturing to pass ratchet protrusion 230. Preferably, the chamfered edge is on the lower side of the O-ring flange. This allows the O-ring flange to move past the barrel opening and ratchet protrusion during assembly of the needle carrier inside the barrel.

The ratchet protrusion 230 at the interior surface of the barrel will therefore restrain the un-chamfered edge at the top of the O-ring flange 90 of the needle carrier from coming out at the proximal end when the plunger is pulled back, as shown in FIG. 13. Hence, the connection means 70 is no longer attached to the needle carrier 120. Therefore, the O-ring flange is prevented from moving any further back up the barrel. In the case of upwards force being used to pull out the plunger, the method by which plunger attaches to the needle carrier allows the plunger and the needle carrier to detach from O-ring flange, preferably due to the elastic and flexible properties of the attachment. The O-ring flange, however, will remain intact and in place 370. The ratchet protrusions 230 are placed low enough within the barrel to make it impossible to pull the O-ring flange out, without damaging the needle carrier. This allows for the needle carrier 120 and the needle 300 to be kept within the barrel. Single or multiple ratchet protrusions can also be used. This feature of the invention allows for single-use of the syringe. Once the needle has been retracted, and the plunger has been pulled out of the barrel at the proximal end the O-ring flange forms a tight seal within the barrel and prevents the needle from being removed from the barrel without breaking barrel.

Before retraction of the needle, at the distal end of the barrel, as shown in FIG. 5, the size of the O-ring flange 90 in relation to the barrel opening allows a tight seal between the outer wall of the O-ring flange 390 and inner wall of barrel 380.

As shown in FIG. 5, the barrel enables the O-ring seat 110 to sit on a shoulder surface 270, and the sidewall near the shoulder is thickened 400. The thickened side wall is sloped to guide the entry of the O-ring as well as the O-ring flange. The sloped surface has 2 functions—it prevents the needle carrier movement if the plunger is depressed by excessive force and it guides the needle tip to fall inside the o-ring seat when needle is push back after retraction. The O-ring now will press against the barrel shoulder rather than on the inner wall of the barrel sleeve. In another preferred embodiment the shoulder is preferably located slightly lower than the compressed thickness of the o-ring.

It is a further advantage of the invention that the dimensions of o-ring are now not as critical as long as the o-ring is wider than opening of sleeve, and smaller than barrel opening. Hence, the usual variations in dimensions of o-ring thickness faced by the prior art, during manufacturing processes is now not an issue. The o-ring can also be used as an effective seal even during the movement of plunger, the top and bottom surfaces always in contact with the o-ring flange and the shoulder of the barrel. Preferably, the o-ring is also under a compressive state regardless its variation in thickness. Hence, various advantages exist over the prior art, including improved sealing properties now makes the syringe leak-proof during operation, reduced number of syringes that are rejected at quality control stage, reduced premature failures of syringes that will not last their shelf life due to lose in elasticity and locking force, reduced risk of product recall, less wastage during production, and more control over manufacturing quality

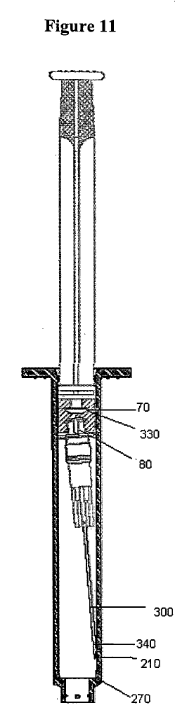

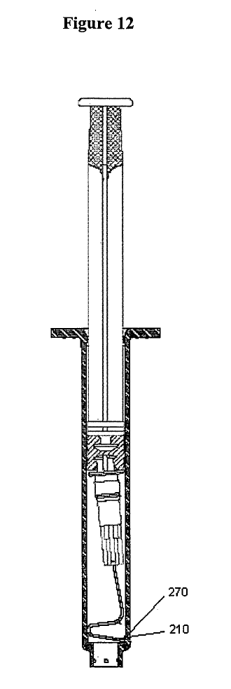

Once the hooks of the needle carrier have been engaged by the connection means and the needle carrier and needle have been retracted into the barrel, and they are canted (FIG. 11), further pushing of the plunger, pushes the needle 300 so that the needle tip 210 is pushed towards the barrel wall 340 and approached to rest on the barrel shoulder 270. The thicker wall at the O-ring side also provides the O-ring with more of a positive sealing. The thickened (or reinforced) barrel wall will bend the needle when the plunger is pushed back after the needle is fully retracted and rest inside the o-ring seat, as shown in FIG. 12, which shows that as the plunger is continually depressed, the needle tip 210 rests on the shoulder 270 and is thereby bent and coiled.

The thicker wall will also reduce the dead space of the syringe because the O-ring flange will match with the wall surface. This is another advantage of the invention over the prior art, which generally shows the overall wall thickness increased to reduce the chances of needle piercing through the barrel. This invention will guide the needle to rest at the o-ring seat before bending commences. Therefore, the wall thickness of the barrel body can be reduced to decrease material usage and to improve the clarity of the syringe.

It is a further advantage of the invention that the sealing device and locking device are separated from each other, so that there is no interaction of both functions. This allows for the O-ring to be compressed in order to obtain sealing without increasing the locking force. The seal is thus maintained even if the O-ring loses part of its elasticity. The locking force will not be affected by reduction in elasticity as the snap lock material does not suffer from change in elasticity with age.

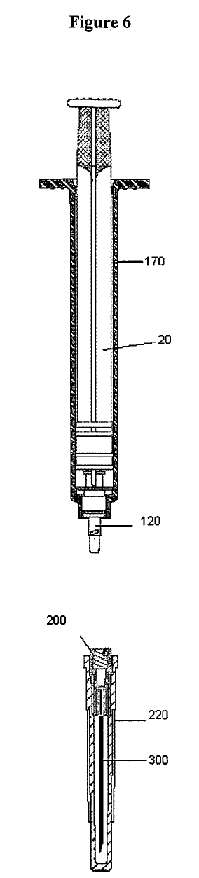

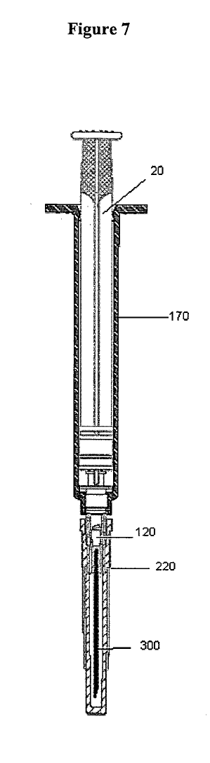

FIG. 1 shows the stem of needle carrier 120, which is attachable to the needle hub 200 as shown in FIG. 6 and FIG. 7. FIG. 7 also shows the needle and needle hub as attached to the needle carrier 120, prior to use of the syringe, whereby the needle and the needle hub are covered by a cap 220. The needle hub is adapted to hold the needle 300. The attachment of the stem of the needle carrier, to the needle hub, is preferably done by using the Luer-lock principle. In anther preferred embodiment of the invention, both the stem and mating hub consist of truncated cones. The stem will enter the recess of the hub first before the screw thread engage. By turning the needle cap together with the needle hub (preferable clockwise), the mating surface of the recess will be in close contact with the surface of the stem and create a seal.

In the final locking position, the diameter of the stem is preferably slightly larger than the diameter of the recess of the hub. Both the stem and the recess should have the same tapered angles. The tip of the stem should not touch the end of the recess, so that the mating surfaces can interface. In another preferred embodiment, the female screw threads can also be single or multi-starts threads.

Standard Luer-lock needles cannot be used in this design because the dimension of the Luer-lock hub is too big to be retracted inside the sleeve and the barrel. Also, the needle can be protruded out of the sleeve if the sleeve opening is too big. Prior art using standard Luer-lock needle requires some device to block the sleeve opening after the retraction of needle. For example, insertion of needle cap or plunger into the sleeve opening. These methods require training and behaviour change and are ineffective for untrained medical professionals.

The stem of needle carrier slides as far into the recess of the hub as the truncated cone allows. At its deepest path, the screw threads engage and the stem is forced to fit with the mating surface. which allows for no leaking from needle base area.

In another preferred embodiment, there are two or more protrusions, which can be shaped triangular on the top (for example, like a pyramid, although variations will be apparent to those skilled in the art), located anywhere on the inner wall of the barrel sleeve.

Preferably, there will be corresponding depressions on needle carrier, which will engage the protrusions, due to shape of protrusions, which will also guide the independently to the direction by which the needle carrier approaches the barrel sleeve. This will allow for a secure connection of the needle carrier and it can be adapted to screw in or out any size needle head, without the needle carrier moving. This has the advantage of providing less risk of injury during needle changing.

The protrusions also regulate the distance between o-ring flange and barrel shoulder, thereby providing a positive compression and sealing before retraction. Therefore, various advantages of the invention are apparent over the prior art, including easy and secure fastening of needles onto needle carrier, improved stock holding and logistics efficiencies. The syringes of the present invention therefore has the same advantages and adaptability usually associated with normal syringes

Preferably, a minimum of two protrusions are within the barrel sleeve, however, there may be a plurality of protrusions. Also, the heads of the protrusions need not be pyramid shaped, but any shape, which allows for the needle carrier and the barrel sleeve to engage securely and yet detach upon retraction.

In a preferred embodiment, the needle carrier material is the same as conventional syringe, that is, medical grade polypropylene. The only non-polypropylene component trapped within the barrel will be the O-ring. Therefore, allowing for lower costs and simpler recycling. The O-ring itself may however, be made with special elastomer that has distinct physical properties from PP. In which case, all the components within the barrel will be recycled easily using their distinct characteristics.

With reference to FIGS. 9 to 13, the operation of the syringe will now be described with particular reference to how the connection means 70 adapts the needle carrier 120 to the plunger 20, and, how the connection means 70 is embodied to effect canting of the needle 300 once it is retracted into the barrel.

Firstly, it should be understood that the connection means is formed using lowest hardness synthetic rubber such as santoprene elastomer. The material must be soft enough to allow the hook to be inserted into the engaging recess easily. The neck of the recess is the same (or slightly smaller) size as the neck of the adaptor. The longitudinal axis of the neck forms an acute angle with the longitudinal axis of the plunger rod. (Theoretically, the longitudinal axis of the plunger rod, the barrel, the needle carrier, and the needle or have the longitudinal axis coincide with each other). The cavity (or chamber) of the engaging recess can be any shape (cylinder, truncated cone and so on) as long as it is bigger than the overall dimension of all the hooks combined.

The hooks 80 of the needle carrier will enter the connection means via the opening. The shape of the opening can be a truncated cone or a radius surface with opening bigger than the outer dimension of the combined hooks. As the hooks enter the engaging recess, the diameter of the opening is progressively smaller until the same size as the neck of the engaging recess. The hooks and the neck of the needle carrier are bent towards centre axis of the needle carrier so that it can pass through the neck of the recess easily. After the hooks have cleared the neck section of the recess, all the hooks will spring back to their uncompressed shape inside the recess chamber.

This configuration has effectively reduced the engaging force during injection, and yet retains the positive locking using the spring back effect of the hooks inside the recess chamber.

By pulling the plunger in the proximal direction, the hooks will engage with the flange inside the enlarged chamber. The v-groove of the needle carrier will slide out the protrusions around the sleeve of the barrel. The locking forces of the protrusions at the sleeve (of the barrel) are always smaller than the engaging forces of the hooks inside the recess chamber. The locking forces can be adjusted by varying the numbers of protrusions, or dimension of protrusions. The needle carrier will be pulled inside the barrel together with the needle. After the needle has cleared the shoulder of the barrel, the neck of the needle carrier will be free to align with the neck of the recess which is inclined at an acute angle with the longitudinal axis of the barrel.

Therefore, the needle, the hub, and the needle carrier are canted inside the barrel.

Further pulling of plunger in proximal direction will pull the needle carrier and needle tip further away from the shoulder of the barrel while the stopper flange of plunger rod will touch the ratchet protrusion and stop the pulling action. The plunger rod is then pushed in the distal direction. The needle tip will touch the sidewall of the barrel until it reaches the corner of the needle tip guide. The tip will slide along the chamfer and the O-ring wall and stop at the corner of the shoulder. Further pushing of the plunger rod will cause the middle length of the needle to bow downward until first bent is formed at one corner of the shoulder. Further pushing will cause the needle to form second bend if the needle is long enough to form a second bend at the other corner. The position of bending and the shape of the coiled needle is very important. The coiled dimension of the needle must be bigger than the distal opening of the barrel to prevent the needle re-protrusion. By keeping the needle tip at the side wall of the barrel, there is no way to expose the sharp after coiling. By pushing the plunger rod, the overall volume of the used syringe will be smaller and reduce the space of the container for safe disposal

The invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

Although the preferred embodiment has been described in detail, it should be understood that various changes, substitutions, and alterations can be made by one of ordinary skill in the art without departing from the scope of the present invention as broadly described hereinbefore and as claimed hereinafter.

1. A disposable syringe, including:
a plunger movable within a barrel;
a needle carrier to retain a needle; and,
a connection means attached to the plunger,
whereby, upon depression of the plunger said connection means is adapted to engage the needle carrier in an angularly displaced manner, such that, upon subsequent withdrawal of the plunger, the needle carrier and needle become housed within the barrel in a canted manner.

2. A disposable syringe as claimed in claim 1, wherein the connection means includes an engaging recess formed with a neck and an enlarged chamber, and, the needle carrier includes a plurality of hooks, each hook having a deformable arm and an enlarged head, such that, when said hooks become fully inserted within the engaging recess, the arms of the hooks spring back to their uncompressed state and the head of each hook is permanently retained within the chamber.

3. A disposable syringe as claimed in claim 1 or 2, wherein said neck of said engaging recess of said connection means is angularly offset from a longitudinal axis of said syringe.

4. A disposable syringe as claimed in any one of claims 1 to 3, wherein said connection means is formed of rubber or like material to permit some deformation during engagement of the needle carrier therewith.

5. A disposable syringe as claimed in any one of claims 1 to 4, wherein each hook is formed of rigid by somewhat flexible plastics or like material, the permit some deformation during engagement with said connection means.

6. A disposable syringe as claimed in any one of claims 1 to 5, wherein said connection means is permanently attached to said plunger.

7. A disposable syringe as claimed in any one of claims 1 to 5, wherein said connection means is removably attached to said plunger.

8. A disposable syringe as claimed in claim 7, wherein said connection means is attached to said plunger via a shaped protrusion being provided on said plunger which is adapted to be received in a corresponding recess provided in said connection means, or vice versa.

9. The disposable syringe as claimed in any one of claims 1 to 8, whereby the needle carrier includes a sealing device as a separate component.

10. The disposable syringe as claimed claim 9, whereby the sealing device includes a flange that is wider than the opening of the barrel, and is adapted to form a sealing between the outer wall of the sealing device and the inner wall of a barrel.

11. The disposable syringe as claimed in claim 10, whereby the sealing device has a seat below the flange.

12. The disposable syringe as claimed in any one of claims 9 to 11, whereby the sealing device is formed as an O-ring.

13. The disposable syringe as claimed in any one of claims 9 to 12, whereby the sealing device has at least one chamfered edge.

14. The disposable syringe as claimed in any one of claims 9 to 13, whereby sealing device is chamfered at the lower side of the sealing device.

15. The disposable syringe as claimed in any one of claims 1 to 14, whereby the barrel has, shoulders at the distal end, which allows for the resting of the sealing device.

16. The disposable syringe as claimed in claim 15, whereby the seat of the sealing device rests on the barrel shoulders.

17. The disposable syringe as claimed in claims 15 or 16, whereby the shoulders are substantially flat with sloped edges.

18. The disposable syringe as claimed in any one of claims 9 to 17, whereby the shoulders are substantially near a thicker barrel wall.

19. The disposable syringe as claimed in any one of claims 1 to 18, whereby the barrel has at least one ratchet protrusion at its proximal end.

20. The disposable syringe as claimed in any one of claims 9 to 19, whereby the chamfered edge of the sealing device allows for the sealing device to pass through the ratchet protrusions of the barrel, upon entering the barrel at the proximal end, however, does not allow for the sealing device to exit the barrel.

21. The disposable syringe as claimed in any one of claims 1 to 20, whereby two or more protrusions are attached to anywhere along the interior barrel wall and/or barrel sleeve for engagement with the needle carrier.

22. The disposable syringe as claimed in claim 21, whereby the protrusions have a pyramid-shaped head.

23. The disposable syringe as claimed in claims 21 or 22, whereby the needle carrier is formed such that it includes depressions, which are able to receive the protrusions along the barrel wall, in order for the barrel to engage the needle carrier.

24. The disposable syringe as claimed in any one of claims 1 to 23, whereby the needle carrier is engaged by the barrel by a locking mechanism.

25. The disposable syringe as claimed in claim 24, whereby the locking mechanism is a snap-lock mechanism.

26. The disposable syringe as claimed in any one of claims 1 to 25, whereby the needle carrier is adapted to hold the needle by the use of a carrier stem.

27. The disposable syringe as claimed in claim 26, whereby the carrier stem is attached to a needle hub.

28. The disposable syringe as claimed in claim 27, whereby the carrier stem and the needle hub are attached using the Luer-lock principle.

29. The disposable syringe as claimed in any one of claims 26 to 28, whereby the stem is formed by a truncated cone.

30. The disposable syringe as claimed in any one of claims 27 to 29, whereby the needle hub is formed by a truncated cone.

31. The disposable syringe as claimed in any one of claims 27 to 30, whereby the stem is inserted into the hub.

32. The disposable syringe as claimed in claim 31, whereby a screw thread engages the stem and hub.

33. The disposable syringe as claimed in claim 31, whereby a female thread engages the stem and hub.

34. The disposable syringe as claimed in any one of claims 27 to 33, whereby the needle hub is turned, thereby creating a seal between the hub and the stem such that the diameter of the stem is substantially larger than the diameter of the hub, and the stem and the hub are at the same angle.

35. The disposable syringe as claimed in any one of claims 1 to 34, whereby the hooks are of spring formation.

36. The disposable syringe as claimed in any one of claims 1 to 35, whereby the connection means is formed of rubber or plastic material.

37. The disposable syringe as claimed in any one of claims 1 to 36, whereby the needle carrier is formed of plastic material.

38. The disposable syringe as claimed in any one of claims 1 to 37, whereby once the needle has been retracted, it is adapted to rest any where along the barrel wall, whereby by further depression of the plunger the needle is bent.

39. The disposable syringe as claimed in any one of claims 1 to 38, whereby once the needle has been retracted, it is adapted to rest any at the shoulders of the barrel wall, whereby by further depression of the plunger the needle pushes the shoulder and is thereby bent.

40. The disposable syringe as claimed in claims 38 to 39, whereby after bending of the needle, further depression of the plunger results in coiling of the needle.

41. A connection means, for use in a syringe, adapted to be attached to a plunger, and to receive a needle carrier, the needle carrier being connected to a needle, such that, the connection means enables a substantial angular displacement of the needle, thereby canting the needle, after the retraction of the needle into the syringe.

42. A connection means as claimed in claim 41, which is formed of rubber or like material to permit some deformation during engagement of the needle carrier therewith.

43. A connection means as claimed in claims 41 or 42, said connection means including an engaging recess formed with a neck and an enlarged chamber, for engagement with a plurality of hooks of said needle carrier, each hook having a deformable arm and an enlarged head, such that, when each hook is inserted within the engaging recess, the arm of the hooks spring back to their uncompressed state and the head of each hook is permanently retained within the chamber.

44. A connection means as claimed in any one of claims 41 to 43, wherein the neck of said engaging recess of said connection means is angularly offset from a longitudinal axis of the syringe.

45. A needle carrier, for use in a syringe, the needle carrier including:
at least one holding means;
a sealing device
whereby the needle carrier is adapted to be attached to a needle, and can also be attached to the plunger of a syringe, through the holding means, and whereby the sealing means is a separate component to the holding means.

46. The needle carrier as claimed in claim 45 whereby the needle carrier is adapted to be attached to a needle, through a stem.

47. The needle carrier, as claimed in any one of claims 45 to 46 whereby the needle holding means includes a plurality of hooks.

48. A sealing device for use in a syringe, the sealing device including a flange, whereby the flange is adapted to be wider than the opening of the syringe, and when inserted into the syringe, the flange is compressed such that to allow sealing between the flange and the syringe.

49. A disposable syringe, substantially as herein described with reference to the accompanying drawings.

50. A disposable syringe, substantially as herein described with reference to the accompanying drawings.
